(19) United States
(12) Patent Application Publication
(10) Pub. No.: US 2007/0202164 A1
(43) Pub. Date: Aug. 30, 2007

(54) Andrographis Extract Formulations

(75) Inventors: Yuqing Wang, Shanghai (CN); Li Wang, Shanghai (CN); Xun Zhang, Shanghai (CN)

Correspondence Address: OCCHIUTI ROHLICEK & TSAO, LLP, 10 FAWCETT STREET, CAMBRIDGE, MA, 02138, US

(73) Assignee: HUTCHISON MEDIPHARMA ENTERPRISES LIMITED

(21) Appl. No.: 11/674,557

(22) Filed: Feb. 13, 2007

Related U.S. Application Data
(60) Provisional application No. 60/777,887, filed on Feb. 28, 2006.

(51) Int. Cl. A61K 36/185; A61K 9/20

(52) U.S. Cl. 424/464; 424/725

(57) ABSTRACT

A pharmaceutical formulation containing 50-90% by weight a powdered extract of Andrographis paniculata and 5-50% by weight a powdered blocking agent. The formulation may further contain a pore-forming agent, a filler, a lubricant, or a glidant. Also described are a method for preparing this pharmaceutical formulation and a method for treating inflammatory disease, immunological disease, or respiratory disease with it.

### CROSS REFERENCE TO RELATED APPLICATIONS

Pursuant to 35 USC § 119(e), this application claims priority to U.S. Provisional Application Ser. No. 60/777,887, filed Feb. 28, 2006, the contents of which are incorporated herein by reference.

### BACKGROUND

Andrographis paniculata (Burm. f.) Nees is a traditional Chinese medicinal herb used to treat diseases such as inflammatory disease, immunological disease, respiratory disease, and cancer. See, e.g., Shen Y. C., et al., Br. J. Pharmacol. 2002, 135(2): 399-406; Rajagopal S. et al., J. Experimental Therapeutics and Oncology, 2003, 3: 147-158; and Wang, et al., China Pharmaceuticals, 2003, 12(10): 72-73. Its major active ingredients are andrographolide, neoandrographolide, 14-deoxy-andrographolide, and 14-deoxy-11,12-didehydroandrographolide.

Extracts of Andrographis paniculata are commercially available as tablets and capsules, which release the active ingredients over a short period of time upon administration. As a result, a high drug concentration in the plasma arises briefly after the administration which causes various side effects such as stomach upset, nausea, and vomiting. Also, a patient needs to take the tablets or capsules frequently in order to keep effective concentrations of the active ingredients in the plasma.

There is a need to develop a pharmaceutical formulation that releases the active ingredients of Andrographis paniculata in a controlled manner.

### SUMMARY

This invention features a pharmaceutical formulation containing 50-90% by weight an Andrographis paniculata extract (“AG extract”) and 5-50% by weight a blocking agent such as hydroxpropyl methylcellulose, acrylic resin, ethyl cellulose, alginic acid, or a mixture thereof. Both the extract and the blocking agent are in the form of powders having sizes in the range of 1-500 μm. Preferably, the extract has powder sizes ranging from 1-180 μm and the blocking agent has powder sizes ranging from 1-160 μm. The pharmaceutical formulation, in contact with water, gradually releases the active ingredients of the AG extract slowly into water, e.g., up to 24 hours. Thus, upon administration of the formulation to a subject, the active ingredients remain at effective concentrations in the plasma for an extended period of time.

The pharmaceutical formulation may additionally contain 0.1-50% by weight a pore-forming agent, which is also in form of powders having powder sizes ranging from 1-500 μm, preferably 1-200 μm. The pore-forming agent can be lactose, starch, microcrystal fibrin, or a mixture thereof. The formulation may also contain 0.1-20% by weight a filler, 0.5-2% by weight a lubricant, or 1-5% by weight a glidant. Examples of a filler include calcium phosphate dibasic, pregelatinized starch, dextrin, calcium sulfate, or a mixture thereof. The lubricant can be magnesium stearate, PEG 4000, or PEG 6000. The glidant, on the other hand, can be French chalk or silicon oxide.

This invention also features a method for preparing the above-described pharmaceutical formulation. The method includes mixing a AG extract powders and blocking agent powders; and aggregating the mixed powders to form granules. Alternatively, the method includes mixing AG extract powders and blocking agent powders with either or both pore-forming agent powders and filler powders; and aggregating the mixed powders to form granules.

The method may include additional steps. For example, after the treating step, the granules are mixed with 0.5-2% by weight a lubricant or 1-5% by weight a glidant, compressed into tablets, or packed in capsules.

This invention further features a method for treating inflammatory, immunological, or respiratory disease by administering to a subject in need thereof the above-described pharmaceutical formulation.

Also within the scope of this invention is use of the above-described pharmaceutical formulation for the manufacture of a medicament for treating inflammatory disease, immunological disease, or respiratory disease.

The details of one or more embodiments of the invention are set forth in the description below. Other features, objects, and advantages of the invention will be apparent from the description, and from the claims.

### DETAILED DESCRIPTION

This invention relates to a pharmaceutical formulation containing 50-90% by weight an AG extract and 5-50% by weight a blocking agent, both of which are in powder form.

To prepare the pharmaceutical formulation, one can first mix AG extract powders with blocking agent powders. The ratio between the extract and the blocking agent is in the range from 1:1 to 18:1.

The AG extract powders preferably contain 7-10% by weight andrographolide, 2-4% by weight neoandrographolide, 0-2% by weight 14-deoxy-andrographolide, and 1-3% by weight 14-deoxy-11,12-didehydroandrographolide. The extract can be produced by extracting Andrographis paniculata with water or an organic solvent (i.e., ethanol or actone), and then removing the water or organic solvent. See, e.g., U.S. patent application Ser. No. 11/116,678. The resultant solid residue is subsequently smashed into powders having powder sizes ranging from 1-500 μm, or, in some embodiments, 1-180 μm. Shown below is an example of how to prepare AG extract powders:

(1) reflux the aerial part of Andrographis paniculata in ethanol of 80° C. for 2 hours,

(2) remove the solid residue and blend the filtrate twice,

(3) conduct the above two steps again except that the solid residue, instead of the aerial part, is used in step (1),

(4) combine and condense all filtrates,

(5) prepare a water solution of dextrin,

(6) combine the condensed solution with the dextrin solution, and

(7) spray-dry the combined solution and smash the dried residue to give powders having a powder size of 1-180 μm.

Once AG extract powders are obtained, it is then mixed with blocking agent powders. The blocking agent has powder sizes ranging from 1-500 μm, or, preferably, 1-160 μm. Examples of a blocking agent include, but are not limited to, hydroxypropyl methylcellulose, acrylic resin, ethyl cellulose, alginic acid, or a mixture thereof. Hydroxpropyl methylcellulose is preferred and two or more types of this polymers can be used together. For example, one can use both hydroxpropyl methylcellulose (K100M) and hydroxypropyl methylcellulose (K15M).

The mixed AG extract powders and blocking agent powders are then caused to bind to form granules, preferably with the aid of mechanical force or a binder (e.g., an aqueous or ethanolic solution of polyvinylpyrrolidone). Granules having powder sizes ranging from 1-1500 μm are preferred can be obtained by sieving.

The granules thus obtained have an unexpected feature, e.g., slowly releasing the AG extract into water. The granules can be further processed into other forms, e.g., tablets or capsules. For example, they can be compressed into tablets, a preferable form.

In a tablet prepared from the granules, the blocking agent serves as a matrix for accommodating the AG extract powders. When contacting water, the blocking agent hydrates and forms a gelatinous barrier layer around the tablet to slow down the release of active ingredients. See, e.g., Rodriguez, C. F. et al., Handbook of Pharmaceutical Controlled Release Technology, Ed., Wise D. L., New York, N.Y.: Marcel Dekker; 2000. The rate of drug release from the tablet matrix depends on the ratio of the blocking agent and the AG extract powders.

To adjust the release rate of the AG extract and enhance the rigidity of the tablet, one can include in the tablet 0.1-50% by weight pore-forming agent powders having powder sizes ranging from 1-500 μm. To do so, one can add pore-forming agent powders to the granules described above. Alternatively, one can mix the pore-forming agent with an AG extract and a blocking agent and then add a binder to the mixture to form granules. Examples of a pore-forming agent include, but are not limited to, lactose, starch, microcrystal fibrin, or a mixture thereof. Lactose is preferred since, in addition to enhancing rigidity, it also avoids disintegration of the tablet.

To provide a bulk volume to a tablet, 0.1-20% by weight a filler can also be added. For examples, one can mix a filler with an extract, a blocking agent, and optionally a pore-forming agent; granulize the mixture; and compress the granules to form a tablet. The filler increases the size of the tablet to facilitate handling.

To further modify tablet properties, a lubricant or a glidant can also be added. As an example, one can mix one or both of these three substances with the granules described above. The resultant mixture is then compressed to form a tablet. A typical tablet contains 0.5-2% by weight a lubricant or 1-5% by weight a glidant.

A lubricant ensures that a tableting powder does not adhere to the equipment used to press the powder during manufacture. It improves the flow of the powder through the presses and minimizes friction and breakage as the finished tablets are ejected from the equipment. A glidant is also used to improve the flowability of a tableting powder during manufacture.

Blocking agents, pore-forming agents, fillers, lubricants, and glidants are all available from commercial sources.

The pharmaceutical formulation, in any of the forms described above (i.e., granules, tablets, or capsules), can be orally administered to treat inflammatory disease or cancer.

Without further elaboration, it is believed that the above description has adequately enabled the present invention. The following specific examples are, therefore, to be construed as merely illustrative, and not limitative of the remainder of the disclosure in any way whatsoever. All of the publications, including the patent application, cited herein are hereby incorporated by reference in their entirety.

### EXAMPLE 1

600 mg of a powered AG extract, 76 mg of calcium phosphate dibasic, 76 mg of hydroxypropyl methylcellulose (K100M), and 76 mg of hydroxypropyl methylcellulose (K15M) were mixed together. The mixture was passed through a 60-mesh sieve (300 μm). A 10% polyvinylpyrrolidone K30 ethanol solution was added to the mixture.

The mixture was passed through a sieve having pores of 1500 μm and after the ethanol had been evaporated, granules having sizes of 1-1500 μm were obtained. The dry granules were mixed with 8.5 mg of silicon oxide and 8.5 mg of magnesium stearate, and the mixture thus obtained was compressed to form a tablet.

The tablet was tested for its rate of releasing active ingredients into 900 ml 0.2% sodium dodecyl sulfate aqueous solution rotated at 75 rpm according to the second method described in the China Pharmacopedia, 2005, Ed: Committee of National Pharmacopedia, pp 73-75. Table 1 below shows the percentages of the AG extract released from the tablet into water over 20 hours.

TABLE 1

| Time (hr) | Percentage of accumulated release (%) |
|---|---|
| 4 | 23.0 |
| 8 | 46.4 |
| 12 | 67.8 |
| 16 | 82.8 |
| 20 | 92.1 |

Release of active ingredients was also tested on a commercially available AG tablet (Yikang pharmaceutical Co., Ltd., Guangdong) and a commercially available AG capsule (Lebang pharmaceutical Co., Ltd., Hunan). Table 2 below shows the percentages of the AG extract released from the tablet and capsule into water over 60 minutes.

TABLE 2

Percentage of
accumulated
release (%)

| Time (min) | Tablet | Capsule |
|---|---|---|
| 10 | 22.7 | 19.6 |
| 20 | 39.2 | 60.5 |
| 30 | 51.6 | 76.1 |
| 45 | 67.9 | 89.1 |
| 60 | 79.4 | 95.0 |

The test shows that it took as long as 16 hours for the tablet to release about 80% the AG extract, and only an hour for the commercially available AG tablet and capsule to release about 80% or more the AG extract.

### EXAMPLE 2

An AG tablet was prepared following a manner similar to that described in Example 1. Table 3 below shows the composition of the tablet.

TABLE 3

| Ingredients | Quantity |
|---|---|
| AG extract powders | 600 mg |
| Calcium phosphate dibasic | 88.2 mg |
| hydroxyl propyl methylcellulose (K100M) | 88.2 mg |
| hydroxyl propyl methylcellulose (K15M) | 88.2 mg |
| silicon oxide | 8.8 mg |
| magnesium stearate | 8.8 mg |

The tablet was tested for its releasing active ingredients into 900 ml 0.2% sodium dodecyl sulfate aqueous solution rotated at 75 rpm according to the second method described in China Pharmacopedia, id. Table 4 below shows the percentages of the AG extract released from the tablet into water over 20 hours.

TABLE 4

| Time (hr) | Percentage of accumulated release (%) |
|---|---|
| 4 | 23.5 |
| 8 | 46.4 |
| 12 | 64.7 |
| 16 | 78.6 |
| 20 | 95.0 |

The test shows that it took more than 20 hours for the tablet gradually release all of the AG extract.

### EXAMPLE 3

An AG tablet was prepared following a manner similar to that described in Example 1. Table 5 below shows the composition of the tablet.

TABLE 5

| Ingredients | Quantity | |
|---|---|---|
| AG extract powders | 600 | mg |
| lactose | 87 | mg |
| hydroxypropyl methylcellulose (K100M) | 70 | mg |
| hydroxypropyl methylcellulose (K15M) | 105 | mg |
| silicon oxide | 8.7 | mg |
| magnesium stearate | 8.7 | mg |

### EXAMPLE 4

An AG tablet was prepared following a manner similar to that described in Example 1. Table 6 below shows the composition of the tablet.

TABLE 6

| Ingredients | Quantity |
|---|---|
| AG extract powders | 600 mg |
| lactose | 120 mg |
| hydroxypropyl methylcellulose | 120 mg |
| (K100M) | |
| microcrystal fibrin | 50 mg |
| silicon oxide | 9 mg |
| PEG 4000 | 9 mg |

### EXAMPLE 5

An AG tablet was prepared following a manner similar to that described in Example 1. Table 7 below shows the composition of the tablet.

TABLE 7

| Ingredients | Quantity |
|---|---|
| AG extract powders | 600 mg |
| starch | 60 mg |
| calcium phosphate dibasic | 60 mg |
| hydroxyl propyl methylcellulose (K15M) | 180 mg |
| silicon oxide | 9 mg |
| magnesium stearate | 9 mg |

### OTHER EMBODIMENTS

A number of embodiments of the invention have been described. Nevertheless, it will be understood that various modifications may be made without departing from the spirit and scope of the invention. Accordingly, other embodiments are also within the scope of the following claims.

What is claimed is:

1. A pharmaceutical formulation comprising 50-90% by weight an Andrographis paniculata extract and 5-50% by weight a blocking agent, wherein the extract and the blocking agent are both in powder form and are uniformly admixed.

2. The formulation of claim 1, wherein each of the extract and the blocking agent, independently, has powder sizes ranging from 1-500 μm.

3. The formulation of claim 2, wherein the extract has powder sizes ranging from 1-180 μm and the blocking agent has powder sizes ranging from 1-160 μm.

4. The formulation of claim 1, further comprising 0.1-50% by weight a pore-forming agent, wherein the pore-forming agent is in powder form.

5. The formulation of claim 4, wherein the pore-forming agent has powder sizes ranging from 1-500 μm.

6. The formulation of claim 4, wherein the blocking agent is 10-20% by weight and the pore forming agent is 5-15% by weight.

7. The formulation of claim 4, wherein the extract includes 7-10% by weight andrographolide, 2-4% by weight neoandrographolide, 0-2% by weight 14-deoxy-andrographolide, and 1-3% by weight 14-deoxy-11,12-didehydroandrographolide; the blocking agent is hydroxypropyl methylcellulose, acrylic resin, ethyl cellulose, alginic acid, or a mixture thereof; and the pore-forming agent is lactose, starch, microcrystal fibrin, or a mixture thereof.

8. The formulation of claim 7, wherein the blocking agent is hydroxypropyl methylcellulose and the pore-forming agent is lactose.

9. The formulation of claim 8, wherein the extract has powder sizes ranging from 1-180 μm, the blocking agent has powder sizes ranging from 1-160 μm, and the pore-forming agent has powder sizes ranging from 1-200 μm.

10. The formulation of claim 1, further comprising 0.1-20% by weight a filler, 0.5-2% by weight a lubricant, or 1-5% by weight a glidant, wherein the filler is calcium phosphate dibasic, pregelatinized starch, dextrin, calcium sulfate, or a mixture thereof; the lubricant is magnesium stearate, PEG 4000, or PEG 6000; and the glidant is French chalk or silicon oxide.

11. The formulation of claim 10, wherein the filler is calcium phosphate dibasic, the lubricant is magnesium stearate, and the glidant is silicon oxide.

12. The formulation of claim 1, wherein the blocking agent is a mixture of hydroxypropyl methylcellulose (K100M) and hydroxypropyl methylcellulose (K15M).

13. A method for preparing a pharmaceutical formulation comprising:
providing a mixture containing a powdered Andrographis paniculata extract and a powdered blocking agent; and
aggregating the mixture to form granules;
wherein the extract and the blocking agent, independently, has powder sizes ranging from 1-500 μm, and the granules have powder sizes ranging from 1-1500 μm.

14. The method of claim 13, further comprising compressing the granules to form a tablet.

15. The method of claim 13, wherein the aggregating step is conducted by adding a binder to the mixture so that the extract and blocking agent powders aggregate to form granules.

16. The method of claim 15, wherein the binder is polyvinylpyrrolidone.

17. The method of claim 13, further comprising mixing a lubricant or a glidant with the granules.

18. The method of claim 17, wherein the lubricant is magnesium stearate, PEG 4000, or PEG 6000 and the glidant is French chalk or silicon oxide.

19. The method of claim 13, wherein the mixture further contains a pore-forming agent in powder form and has powder sizes ranging from of 1-500 μm.

20. The method of claim 19, wherein the pore forming agent is lactose, starch, microcrystal fibrin, or a mixture thereof.

21. The method of claim 13, wherein the mixture further contains a filler, wherein the filler is calcium phosphate dibasic, pregelatinized starch, dextrin, calcium sulfate, or a mixture thereof.

22. The method of claim 21, wherein the mixture further contains a filler, wherein the filler is calcium phosphate dibasic, pregelatinized starch, dextrin, calcium sulfate, or a mixture thereof.

23. The method of claim 22, further comprising mixing a lubricant or a glidant with the granules.

24. The method of claim 23, wherein the lubricant is magnesium stearate, PEG 4000 or PEG 6000; and the glidant is French chalk or silicon oxide.

25. The method of claim 24, further comprising compressing the granules to form a tablet.
